SEQUENCE LISTING (26 sequences)

SEQ ID NO: 1 - DNA, 1681 bp, Capsicum annuum cv. Bukang
accagtttagttgatacttctttggatctcagcttcagacctcttcgagttcctgatgatataccggtgtgtggagatctataaaactattttacagttgattatttctcatatcatcgcttaactaatagtacatgttagctcagaaagtcacgatttaatttctttgtcgaagaaaattggaatcaggaaggaggatcgtgattttccactgtataatcaatcatatgcatgaattagctagtttgccttaatgttttaatttgatttgcttttgctctcagaaacaagaagttgagagtaatttcattgggcttggaagagatctcatgcctgacaaagatgatcaggcaggtgatttgttggaggaactaaatagagtaagtgctgaaaacaagaaactgactgagatgttaacagttatgtgccagaattacaatgcattgagaaaccaattgactgaatatttgaacaagcaaaacagtactactagtactgcagctgataataatcatgatcatcatagcgatggatcgaaaaaaagaaaagtcgaaaacaacaacaatgaaattgtgaaatcagttcaaggattacactcagagagcagctcaagtgatgaagattcatctaacaagaaaccaagagaacaacacattaaaactaacacttgtagagtttatgtcaaaaccgaagcatctgatacttctcttattgtgaaggatggatatcagtggaggaaatatggtcagaaagtaacaagagataacccatctcccagagcttatttcaaatgctcttttgctcctacctgcccagtcaaaaaaaaggtgcaaagaagtgtggaagaccaatcgattctagtagcgacatatgaaggagaacacaaccattccaaaatggatggttcaggccccgttacaacttccccgtctagccgattaaacccgaaaaatactcttgtgggtgctaatactactactgtcatgccatgctccagtactagtatcatcaacacaccatcaggaccaaccttaacattggatcttacacaaccaaaaaaattacaaaatgaccaaaagaaagtgaacagcaatactagtactagtaatgcaagtggtcaaaaaagcaaatcaccaggaggacatgatcatcatcagcaaaatagaccagagtttcaacagttgtttatagatcaaatggcttcttcattgactaaagatccaagttttcaagcagccttagccgctgccatatcgggaaaattcttacaaaataatcatacagacaaataaatgatgattataatcaaactgtgaacttcattgttaatgagctgttcaatgtttttcaagttgacacatttgagaaatgttccagaaaaagatatatagccactaggatatgttgtcaagatgtaaataatgttcatttttggtgaacggtacgtaggttggactaggaagtgcacgtggaaatggaatgggctaattttctatggcagcctaaattggttaggtctcaatttgtgcctatgagtctcacaagattgtgacttgggtgggccttaaattgttgaatttgctcatttatgcaatttgcgaaaataaatacttataatagtaaaaaaaaaaaaaaaaaaaaaaaaaaaaaaaaaaaaaaaaaaaaaaaaaaaaaaaaaaaa

SEQ ID NO: 2 - protein, 320 aa, Capsicum annuum cv. Bukang
MPDKDDQAGDLLEELNRVSAENKKLTEMLTVMCQNYNALRNQLTEYLNKQNSTTSTAADNNHDHHSDGSKKRKVENNNNEIVKSVQGLHSESSSSDEDSSNKKPREQHIKTNTCRVYVKTEASDTSLIVKDGYQWRKYGQKVTRDNPSPRAYFKCSFAPTCPVKKKVQRSVEDQSILVATYEGEHNHSKMDGSGPVTTSPSSRLNPKNTLVGANTTTVMPCSSTSIINTPSGPTLTLDLTQPKKLQNDQKKVNSNTSTSNASGQKSKSPGGHDHHQQNRPEFQQLFIDQMASSLTKDPSFQAALAAAISGKFLQNNHTDK

SEQ ID NO: 3 - DNA, 21 bp, Artificial Sequence
  PCR PRIMER
caagttttcaagcagccttag

SEQ ID NO: 4 - DNA, 24 bp, Artificial Sequence
  PCR PRIMER
cttgacaacatatcctagtggcta

SEQ ID NO: 5 - DNA, 20 bp, Artificial Sequence
  PCR PRIMER
gaggacaacgtccgtatggt

SEQ ID NO: 6 - DNA, 24 bp, Artificial Sequence
  PCR PRIMER
aactccagttactgcaccattaga

SEQ ID NO: 7 - DNA, 24 bp, Artificial Sequence
  PCR PRIMER
ctacttaagctttgcaagacacca

SEQ ID NO: 8 - DNA, 24 bp, Artificial Sequence
  PCR PRIMER
agatctctttcctcatcgtcactt

SEQ ID NO: 9 - DNA, 20 bp, Artificial Sequence
  PCR PRIMER
gaacacaagcaacggtgaga

SEQ ID NO: 10 - DNA, 20 bp, Artificial Sequence
  PCR PRIMER
ggcacttgtttaggcagagc

SEQ ID NO: 11 - DNA, 24 bp, Artificial Sequence
  PCR PRIMER
ttgccaaagttggactactgatta

SEQ ID NO: 12 - DNA, 24 bp, Artificial Sequence
  PCR PRIMER
tcaaccaaattgaacaaaaagaga

SEQ ID NO: 13 - DNA, 20 bp, Artificial Sequence
  PCR PRIMER
gactagtttcaagagcatca

SEQ ID NO: 14 - DNA, 20 bp, Artificial Sequence
  PCR PRIMER
aattgtatagcctgtagctg

SEQ ID NO: 15 - DNA, 24 bp, Artificial Sequence
  PCR PRIMER
ctgtctatgtttaagccaatgacc

SEQ ID NO: 16 - DNA, 24 bp, Artificial Sequence
  PCR PRIMER
aaagttctttccatgacaaccaat

SEQ ID NO: 17 - DNA, 20 bp, Artificial Sequence
  PCR PRIMER
ctgtaaagagctcacaaaac

SEQ ID NO: 18 - DNA, 19 bp, Artificial Sequence
  PCR PRIMER
gcgagagttttagcattac

SEQ ID NO: 19 - DNA, 20 bp, Artificial Sequence
  PCR PRIMER
ccctagaggacttgtacatt

SEQ ID NO: 20 - DNA, 20 bp, Artificial Sequence
  PCR PRIMER
ttagtcaagacagaatcagg

SEQ ID NO: 21 - DNA, 20 bp, Artificial Sequence
  PCR PRIMER
cttgtctgtgataatggaac

SEQ ID NO: 22 - DNA, 21 bp, Artificial Sequence
  PCR PRIMER
ggatacttcaaggtgaagaat

SEQ ID NO: 23 - DNA, 23 bp, Artificial Sequence
  PCR PRIMER
ggatccaaatgttccagaaaaag

SEQ ID NO: 24 - DNA, 23 bp, Artificial Sequence
  PCR PRIMER
ggatccaggcccacccaagtcac

SEQ ID NO: 25 - DNA, 20 bp, Artificial Sequence
  PCR PRIMER
cccgggcaatgcctgacaaa

SEQ ID NO: 26 - DNA, 22 bp, Artificial Sequence
  PCR PRIMER
cccgggtttgtctgtatgatta

(19) United States
(12) Patent Application Publication
(10) Pub. No.: US 2011/0321201 A1
(43) Pub. Date: Dec. 29, 2011

(54) CaWRKYd, A HOT PEPPER TRANSCRIPTION FACTOR GENE INVOLVED IN PLANT DEFENSE AND TRANSGENIC PLANTS USING THE SAME

(75) Inventors: Kyung-Hee PAEK, Seoul (KR); La Mee CHOI, Seoul (KR); Gil-Je LEE, Seoul (KR); Jee Hyuck LIM, Sungnam (KR)

(73) Assignee: KOREA UNIVERSITY RESEARCH AND BUSINESS FOUNDATION, Seoul (KR)

(21) Appl. No.: 13/085,888

(22) Filed: Apr. 13, 2011

(30) Foreign Application Priority Data
Jun. 28, 2010 (KR) 10-2010-0060964

(51) Int. Cl. A01H 5/00; C07K 14/415; C12N 15/82; C07H 21/04

(52) U.S. Cl. 800/298; 536/23.6; 530/379; 435/320.1

(57) ABSTRACT

Disclosed herein is the novel plant-specific transcription factor gene CaWRKYd that can be induced by interaction with TMV-P₀ and that plays an important role in regulating plant defense responses through a CaMK1 cascade induced and acts as a positive regulator imparting disease resistance to plants. Also, a transgenic plant anchoring the gene therein is provided. Therefore, the gene allows the production of plants resistant to diseases and can be very effectively used in studying defense response mechanisms of plants.

### BACKGROUND OF THE INVENTION

1. Field of the Invention

The present invention relates to a CaWRKYd gene, a plant-specific transcription factor gene involved in the defense response of hot pepper plants, and a transgenic plant prepared using the same. More particularly, the present invention relates to a plant-specific transcription factor gene, identified as CaWRKYd, which plays an important role in regulating TMV-P₀-induced plant defense responses by interacting with CaMK1 and functions as a positive regulator to give disease resistance to plants, and a transgenic plant anchoring the same therein.

2. Description of the Related Art

Host plants, when infected with pathogens, operate defense mechanisms in various intracellular metabolic pathways. Interactions between host plants and pathogens are divided into compatible or incompatible interactions in the early stages of infection. Depending on the interactions, it is determined whether the host plants trigger resistant (defense) reactions to restrict the progress of the disease or not.

In such incompatible interactions, the infected plant recognizes the invasion of pathogens, with the subsequent occurrence of subsequent defense responses actions including hypersensitive responses, such as local cell death at the infected region, callose deposition, and cell wall thickening by lignification. Also, the plant produces various antimicrobial substances including phytoalexins and synthesizes pathogenesis related (PR) proteins to exert responses that resist the infection.

These defense mechanisms of plants can be induced by the stimuli of plant hormones such as salicylic acid and methyl jasmonate, as well as the invasion of pathogens. Also, environmental stresses such as physical wound, salts and temperature are reported to trigger the activation of plant defense mechanisms.

The expression of plant disease resistance by interactions between host plants and pathogens, and its related signal pathways play an important role in inducing defensive reactions to pathogens among the defensive reactions to biological/physical/environmental stress. How plant disease defense mechanisms and genes involved in resistant responses recognize pathogens and are transmitted over the entire plant has been an important subject of research. In this regard, the functions of disease defense-related (resistant) genes have been studied using biological, biochemical, and cytobiological methods.

A typical trait of plant defense reactions is to activate the transcription of various genes upon pathogen infection or treatment with pathogen elicitors (Rushton and Somssich, Curr. Opin. Plant Biol, 1998; Yang et al., Genes Dev, 1997). Some of the genes whose expression is induced by pathogens encode proteins that have inhibitory activity against microbes or enzymes involved in the biosynthesis of antimicrobial compounds. Other pathogen-inducible genes encode proteins that regulate signal pathways of plant defense reactions. A change in the expression of specific transcription factors has a great influence on the plants under stress.

Among the several classes of transcription factors associated with plant defense responses are the recently identified DNA-binding proteins containing WRKY domains that appear to be prevalent in plants (Eulgem et al. Trends Plant Sci, 2000). The WRKY domains contain a conserved WRKYGQK sequence followed by a Cys2His2 or Cys2HisCys zinc-binding motif. WRKY transcription factors are encoded by a multigene family comprising over 75 members in Arabidopsis (Eulgem et al, Trends Plant Sci, 2000). In addition to their DNA-binding ability, WRKY proteins share other features of transcription factors, such as nuclear localization and transcription activation capability (Hara et al., Mol Gen Genet, 2000). A number of studies have shown that WRKY proteins probably have regulatory functions in plant defense responses to pathogen infection. First, pathogen infection or treatment with pathogen elicitors or SA has been shown to induce rapid expression of some WRKY genes from a number of plants (Chen and Chen, Plant Mol Biol, 2000; Dellagi et al., Mol Plant Microbe Interact, 2000; Eulgem et al., Embo J, 1999; Hara et al., Mol Gen Genet, 2000; Kim et al., Mol Plant Microbe Interact, 2000; Rushton et al., EMBO J, 1996). Secondly, a number of defense-related genes, including PR genes, contain W-box elements in their promoter regions (Du and Chen, Plant J, 2000; Eulgem et al., EMBO J, 1999; Rushton et al., EMBO J, 1996). In addition, W-box sequences are specifically recognized by WRKY proteins and are necessary for the inducible expression of these genes (Du and Chen, Plant J, 2000; Eulgem et al., EMBO J, 1999; Rushton et al., EMBO J, 1996).

Increasing studies about the WRKY proteins have been reported during past 15 years. However, most of these findings are restricted to only a few plants including Arabidopsis, rice, parsley, or tobacco (Chen and Chen, Plant Mol Biol, 2000; Eulgem et al., Trends Plant Sci, 2000; Rushton et al., Embo J, 1996; Zhang et al., Plant Physiol, 2004).

The present inventors isolated a CaWRKY-a as the first WRKY transcription factor gene from hot pepper and found that this transcription factor was specifically induced during a hypersensitive interaction between C. annuum cv. Bugang and TMV-Po and exhibited responses to varieties of stress including biotic or abiotic elicitors and wounding, demonstrating that the protein functions as a transcription factor in defense-related signaling pathway of plants (Korean Patent No. 10-0789846). Other WRKY transcription factors derived from hot pepper include CaWRKY1 (Oh et al., New Phytologist 2008) and CaWRKY2 (Oh et al., Mol. Cells, 2006).

Hot pepper (Capsicum annuum L.) is a representative member of the nightshade family, Solanaceae. The plant is native to South America and is cultivated widely over the tropical and temperate regions. After being introduced into Korea, although unknown at the beginning of its introduction, hot pepper has had a great influence on the dietary habits of Koreans. Particularly, hot pepper is one of the most abundant garden products in Korea and is very important in the agricultural economy. Various diseases of hot pepper plants, caused by viruses, bacteria, and fungi, have been reported. In practice, the annual production of hot pepper is greatly influenced by the incidence of disease. However, no effective disease-resistant hot pepper plants have been reported to date. In fact, protecting the plants from diseases is, for the most part, dependent on chemical control. Therefore, the isolation of a defense-related gene and the production of transgenic plants using bioengineering techniques, which overcome the drawbacks of the conventional breeding techniques directed toward the development of newly resistant plants, reduces the production cost which increases the income of farmers, and enables the production of quality clean crops in line with the policy of being friendly to the environment, thus contributing to the activation of various related industries.

### SUMMARY OF THE INVENTION

Leading to the present invention, intensive and thorough research into plant defense systems, conducted by the present inventors, resulted in the finding that CaWRKYd interacts with CaMK1 and functions as a positive regulator in TMV-P₀-induced defense responses, hence identifying CaWRKYd as a plant-specific transcription factor gene involved in the defense system of hot pepper.

It is therefore an object of the present invention to provide a gene CaWRKYd encoding a plant-specific transcription factor involved in hot pepper plant defense, and a protein encoded thereby.

It is another object of the present invention to provide a recombinant vector carrying the gene or a vector for inhibiting the expression of the gene.

It is a further object of the present invention to provide a transgenic plant, established by introducing the CaWRKYd gene into a plant, which is resistant to diseases.

In accordance with an aspect thereof, the present invention provides an isolated gene encoding a hot pepper-derived stress-resistant transcription factor having the amino acid sequence of SEQ ID NO. 2.

In an embodiment, the gene has a nucleotide sequence of SEQ ID NO. 1.

In accordance with another aspect thereof, the present invention provides an isolated protein having the amino acid sequence of SEQ ID NO. 2.

In accordance with a further aspect thereof, the present invention provides a recombinant vector carrying the gene.

In accordance with still a further aspect thereof, the present invention provides a transgenic plant anchoring the gene or the recombinant vector.

In accordance with still another aspect thereof, the present invention provides a pTRV2-CaWRKYd vector for VIGS (virus induced gene silencing), constructed by cloning a hot pepper transcription factor gene CaWRKYd having the nucleotide sequence of SEQ ID NO. 1 into a TRV vector.

In accordance with yet another aspect thereof, the present invention provides a transgenic plant in which a CaWRKYd gene is down-regulated, the transgenic plant anchoring a pTRV2-CaWRKYd vector for VIGS (virus induced gene silencing) which is constructed by cloning a hot pepper transcription factor gene CaWRKYd having the nucleotide sequence of SEQ ID NO. 1 into a TRV vector.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing the nucleotide sequence of the CaWRKYd gene of the present invention, along with its amino acid sequence;

FIG. 2 is a phylogenetic tree showing the predicted evolutionary relationship of CaWRKYd with other WRKYs, based on their amino acid sequence similarity, in which the horizontal line length is an indication of the dissimilarity between the sequences they connect and the numbers below the line indicate the relative distance from the near node;

FIG. 3 is a view showing the comparison of an amino acid sequence deduced from the CaWRKYd cDNA of the present invention with WRKYs from other species, sequences of WRKYs from Capsicum annuum (CaWRKYd), Solanum lycopersicum (CAN 38396), Larrea tridentate (AAW30662), Capsicum annuum (AAX20040), Arabidopsis thaliana (AtWRKY40, NP178199), and Boea hygrometrica (AC162177);

FIG. 4 shows the expression analysis of CaWRKYd upon inoculation with TMV-P₀ by qRT-PCR. Total RNA was extracted from leaves at 0, 12, 24, 48, 72 hrs after inoculation (HAI). As a control, leaves were mock-inoculated with buffer and carborundum only. Values indicate mRNA levels normalized to the expression of the CaActin gene;

FIG. 5 shows the silencing of the CaWRKYd gene in chili pepper (Capsicum annuum) plants. Two-week-old chili pepper seedlings were inoculated with the pTRV1 and pTRV2-CaWRKYd vectors by agroinfiltration. Four weeks later, upper leaves were challenged with TMV-P₀. Photographs show the leaves of pTRV2 and pTRV2-CaWRKYd 4 days after inoculation with TMV-P₀;

FIG. 6 shows the differential expression of the Capsicum annuum pathogenesis-related (CaPR) genes upon inoculation with TMV-P₀ in CaWRKYd-silenced plants. Expression of CaPR genes in the leaves of pTRV2 and pTRV2-CaWRKYd silenced plants was analyzed by qRT-PCR. Total RNA was extracted from leaves 24, 48, 72 and 96 h after TMV-P₀ inoculation. Values indicate mRNA levels normalized to the expression of the CaActin gene;

FIGS. 7A to 7D show a qRT-PCR analysis of the expression of CaWRKYd after the induction of Capsicum annuum CaWRKYd by defense signaling molecules and wounding treatment;

FIG. 7A shows that transcripts of CaWRKYd were detected in chili pepper plants (Capsicum annuum cv. Bukang) treated with salicylic acid (SA). Hot pepper leaves were sprayed with 10 mM SA or mock-treated (10% ethanol);

FIG. 7B shows that transcripts of CaWRKYd were detected in chili pepper plants (Capsicum annuum cv. Bukang) treated with ethephon. Hot pepper leaves were sprayed with 10 mM ethephon or mock-treated (10% ethanol);

FIG. 7C shows that transcripts of CaWRKYd were detected in chili pepper plants (Capsicum annuum cv. Bukang) treated with MeJA. Hot pepper leaves were sprayed with 100 μM MeJA or mock-treated (10% ethanol);

FIG. 7D shows that transcripts of CaWRKYd were detected in chili pepper plants (Capsicum annuum cv. Bukang) wounded at various time-points. Wounds were made on the leaves of chili pepper plants by crushing the apical lamina several times, resulting in effectively wounding ˜90% of the leaf area. Values indicate mRNA levels normalized to the expression of the CaActin gene;

FIG. 8 shows qRT-PCR analysis of the tissue-specific expression of CaWRKYd in various tissues of chili pepper plants. Total RNA extracted from various tissues was used. Values indicate mRNA levels normalized to the expression of the CaActin gene;

FIGS. 9A to 9B show the subcellular localization of GFP3G::CaWRKYd fusion protein;

FIG. 9A is a schematic view showing the structure of the GFP3G::CaWRKYd fusion gene; and

FIG. 9B shows the subcellular localization of GFP3G CaWRKYd fusion protein. The GFP3G::CaWRKYd fusion construct was introduced into Arabidopsis protoplast by PEG-mediated transformation. The expression of the introduced genes was monitored 24 hrs after the introduction using fluorescence or light microscopy.

### DESCRIPTION OF THE PREFERRED EMBODIMENTS

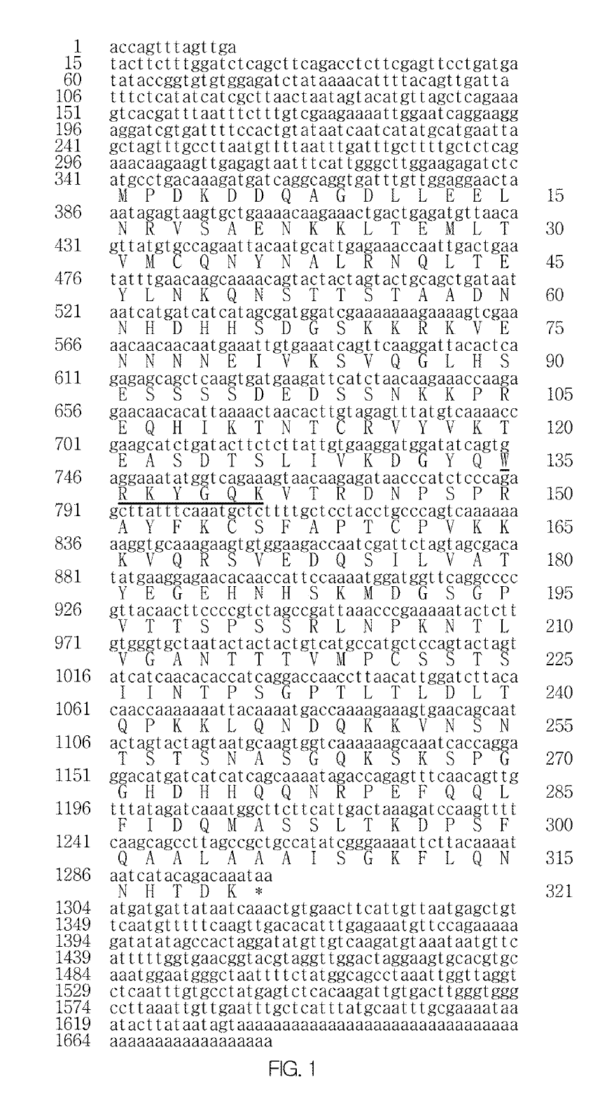

The present invention pertains to the CaWRKYd gene represented by SEQ ID NO. 1, or homologs and variants thereof. Also, the present invention pertains to the CaWRKYd protein represented by SEQ ID NO. 2, or variants thereof. The term “homologs” of the CaWRKYd gene, as used herein, is intended to refer to genes comprising nucleotide sequences encoding CaWRKyd protein comprising the amino acid sequence of SEQ ID NO. 2. Accordingly, all genes that encode the CaWRKYd protein having the amino acid sequence of SEQ ID NO. 2 fall within the scope of the homologs. The term “variants” of the CaWRKYd protein, as used herein, is intended to refer to mutants different from the wild-type CaWRKYd protein in at least one amino acid residue as a result of deletion, addition or substitution. The term “variants” of the CaWRKYd gene refers to all genes encoding the variants of the CaWRKYd protein. Preferably, the gene of the CaWRKYd protein has the nucleotide sequence of SEQ ID NO. 1. Also, it is preferred that the protein has the amino acid sequence of SEQ ID NO. 2 (FIG. 1).

WRKY transcription factors, appearing to be restricted to the plant kingdom, play an important role in regulating pathogen-induced defense responses. Capsicum annuum WRKYd (CaWRKYd) was identified to encode a putative substrate of the Capsicum annuum MAP kinase 1 (CaMK1) from yeast two-hybrid screening of hot pepper EST clones.

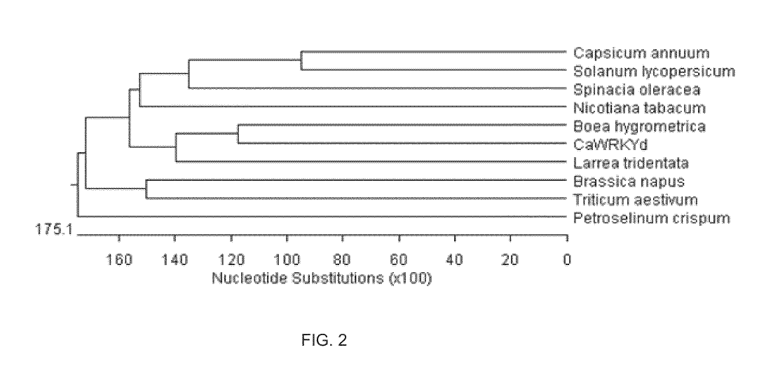

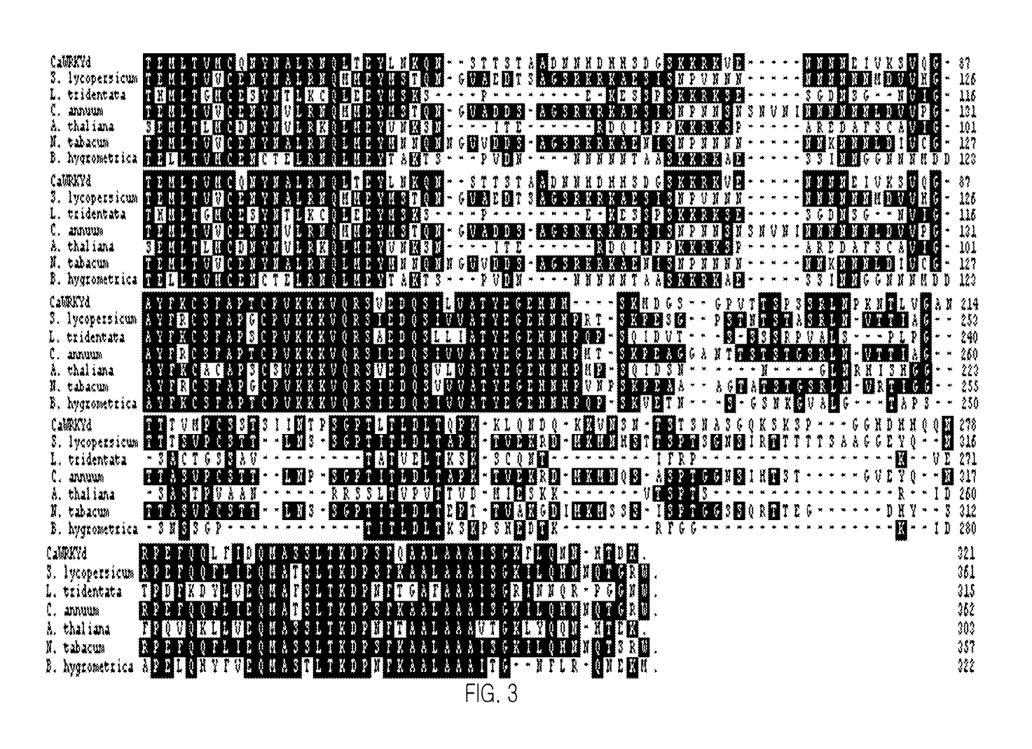

CaWRKYd belongs to WRKY group IIa that has a single WRKY domain and a Cys₂-His₂ zinc-finger motif. CaWRKYd contains a single open reading frame of 963 bp encoding 320 amino acid residues and was found to be localized in the nucleus according to GFP-fused protein targeting experiments. The amino acid sequence was compared with WRKY sequences derived from various plant species (FIGS. 2 and 3). Many studies show that WRKY has regulatory functions related to the defensive response of plants to infection by pathogens (Chen and Chen, Plant Mol Biol, 2000; Dellagi et al., Mol Plant Microbe Interact, 2000; Eulgem et al., Trends Plant Sci, 2000; Kim et al., Mol Plant Microbe Interact, 2000; Rushton et al., EMBO J, 1996; Du and Chen, Plant J, 2000). It is inferred from these previous studies that the CaWRKYd protein of the present invention functions as an inducible transcription factor resistant to stress.

To investigate whether the CaWRKYd gene of the present invention is involved in disease resistance, the expression of the gene in response to various biotic or abiotic types of stress was measured.

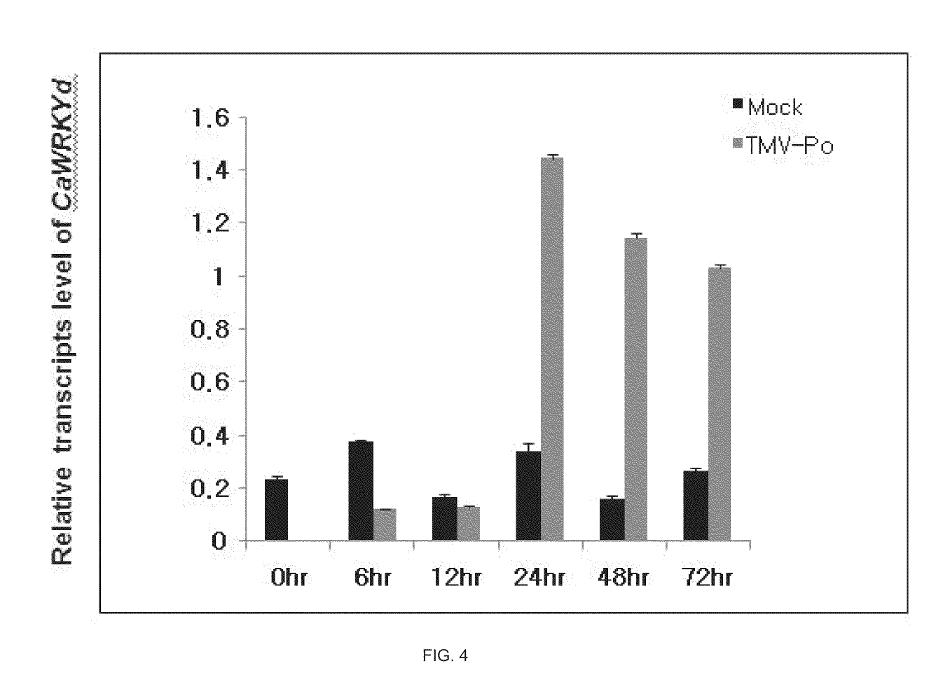

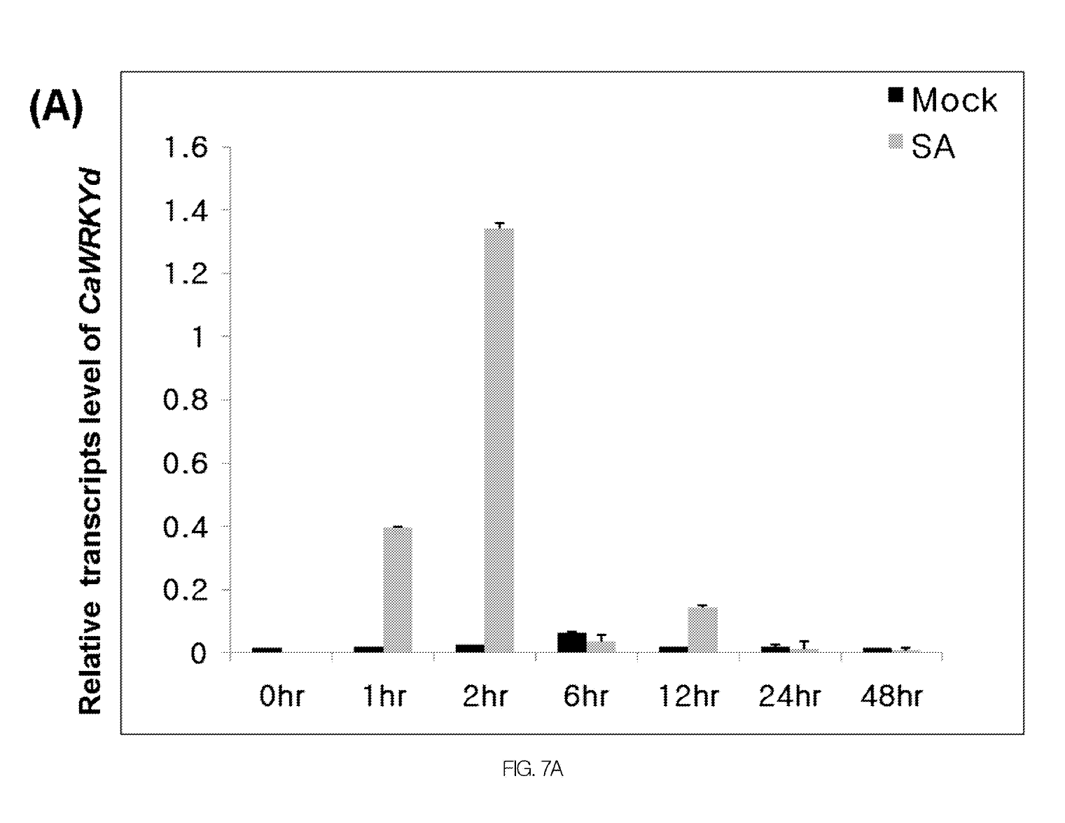

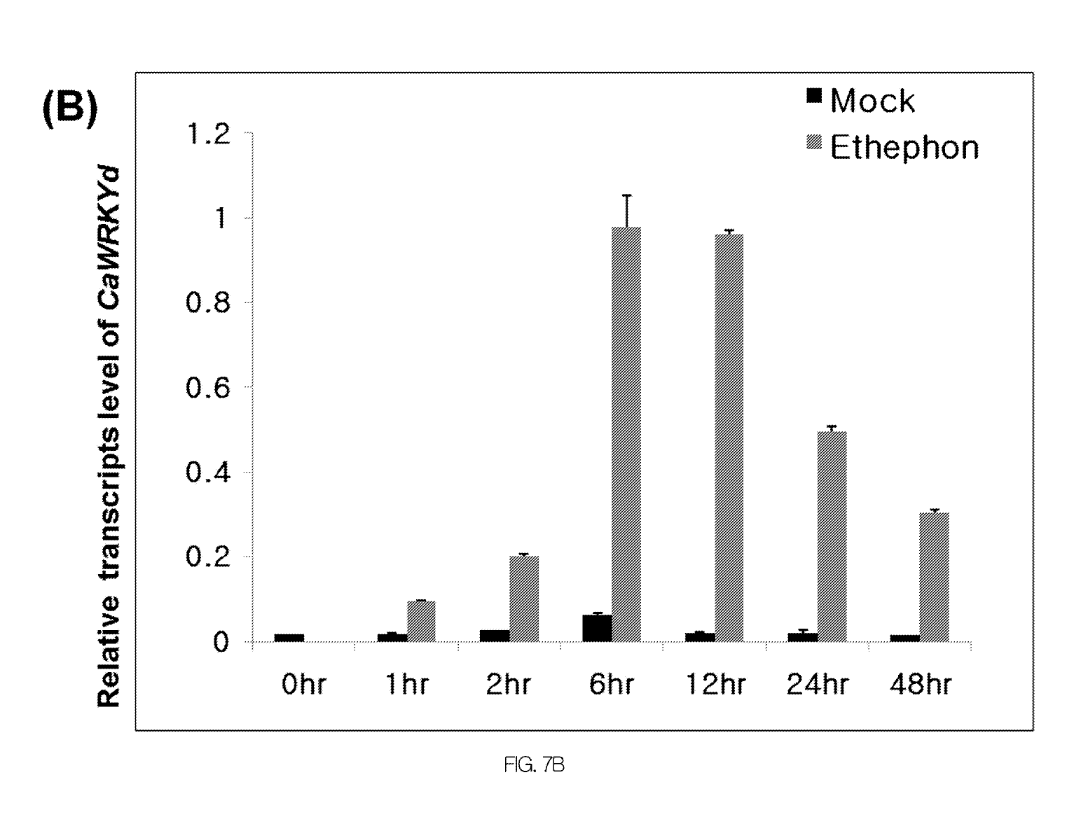

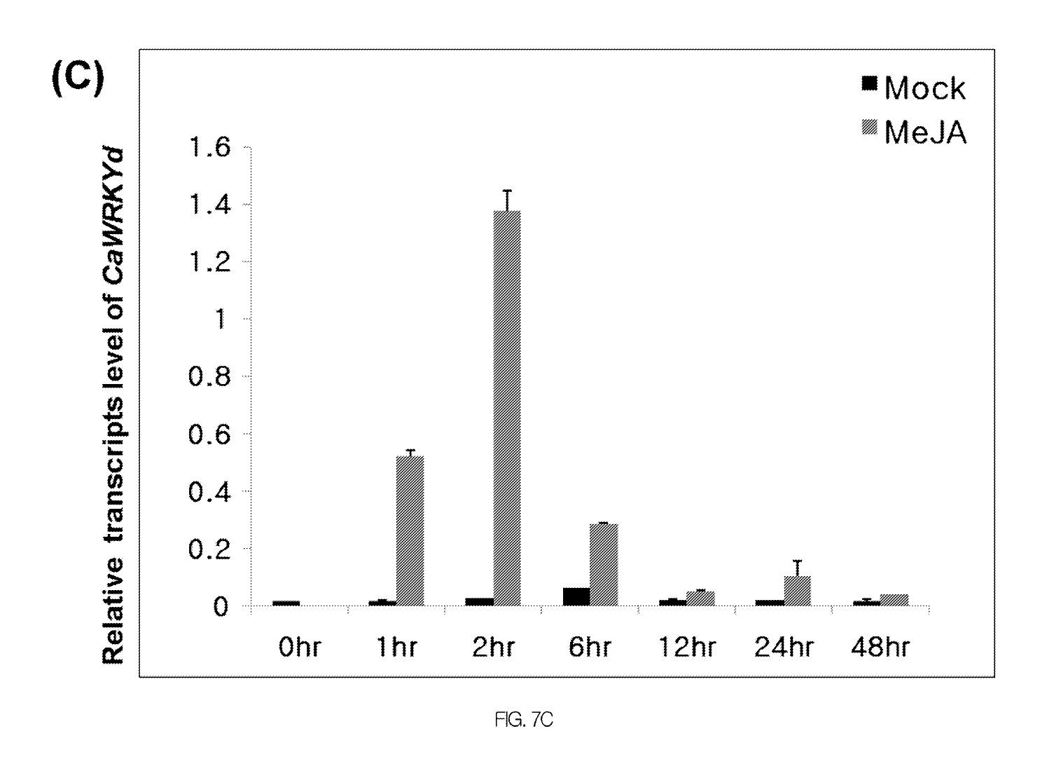

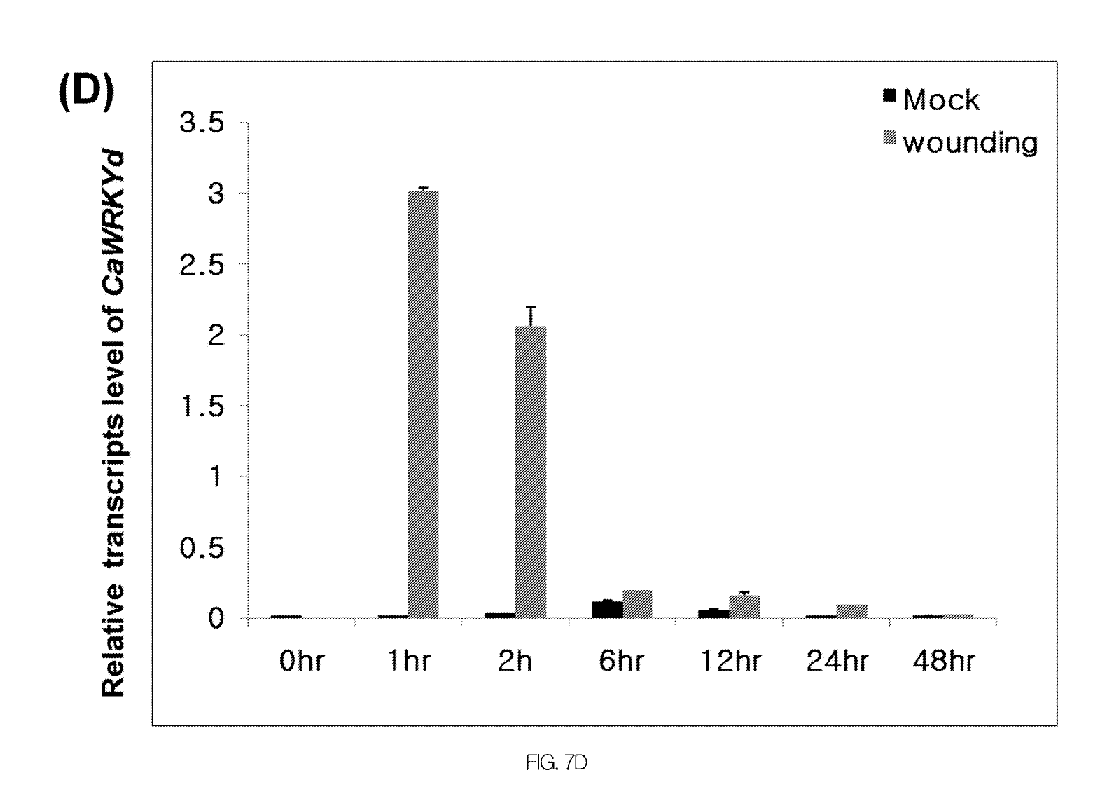

First, inoculation with TMV-P₀ increased the expression level of CaWRKYd in hot pepper (FIG. 4). To investigate whether CaWRKYd was also induced by other stimuli, plants were treated with salicylic acid, ethephon, and methyl jasmonic acid (MeJA), all known to function as signal molecules in defense-related signaling pathways. CaWRKYd was rapidly induced by SA and MeJA treatment, and also induced by ethephon (FIGS. 7A to 7C) and wounding treatment (FIG. 7D).

These results indicate that CaWRKYd is induced by various types of stress and functions as an important factor in plant defense responses.

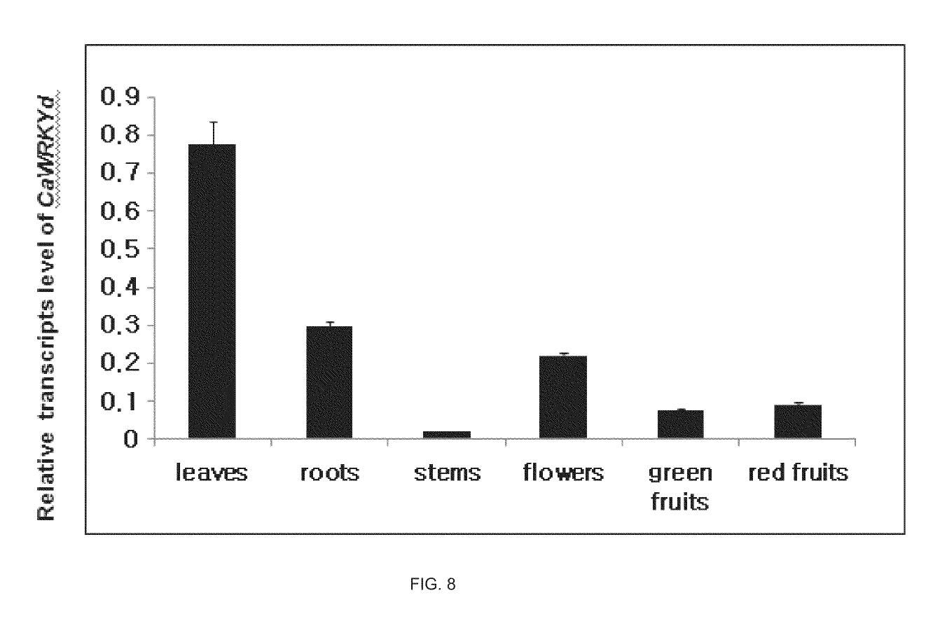

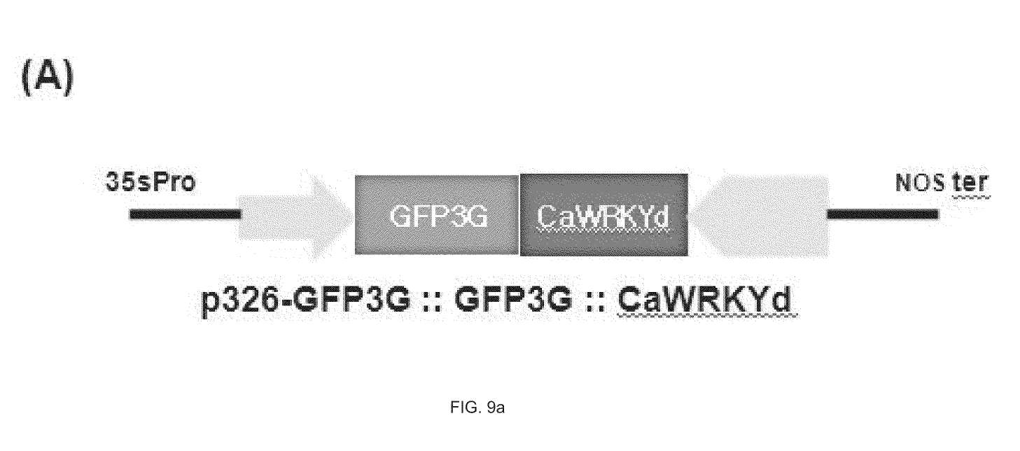

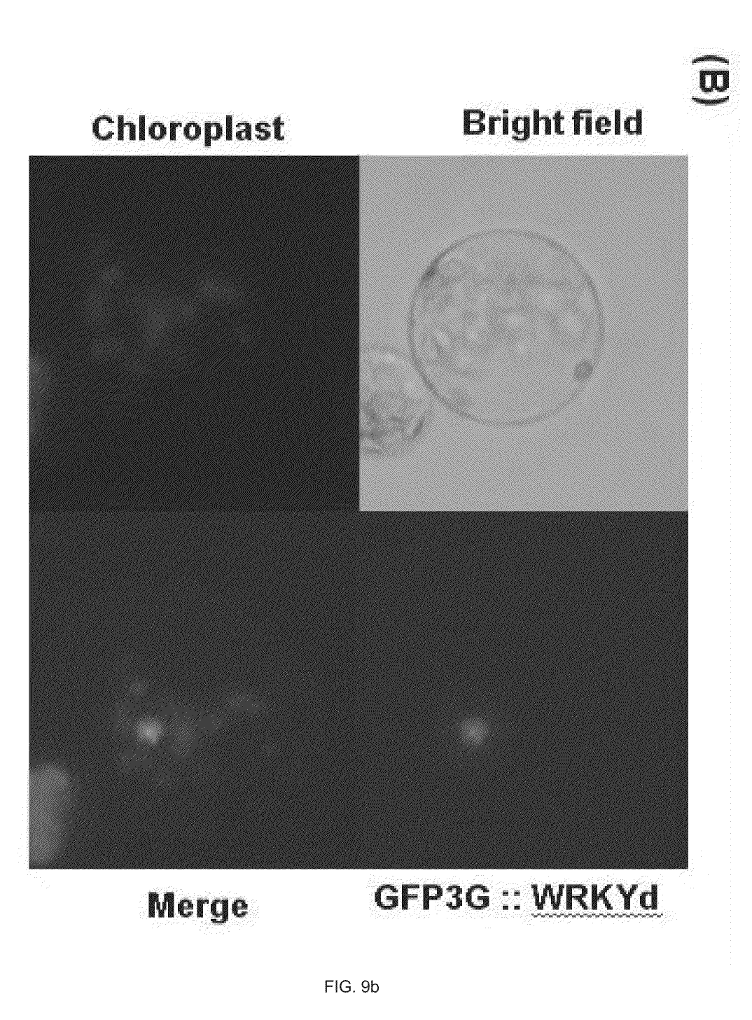

CaWRKYd transcripts were detected in all organ tissues including the leaves, roots, stems, flowers and fruit flesh, and were especially abundant in the leaves (FIG. 8). GFP-fused protein targeting experiments showed the nuclear localization of the CaWRKYd (FIGS. 9A to 9B).

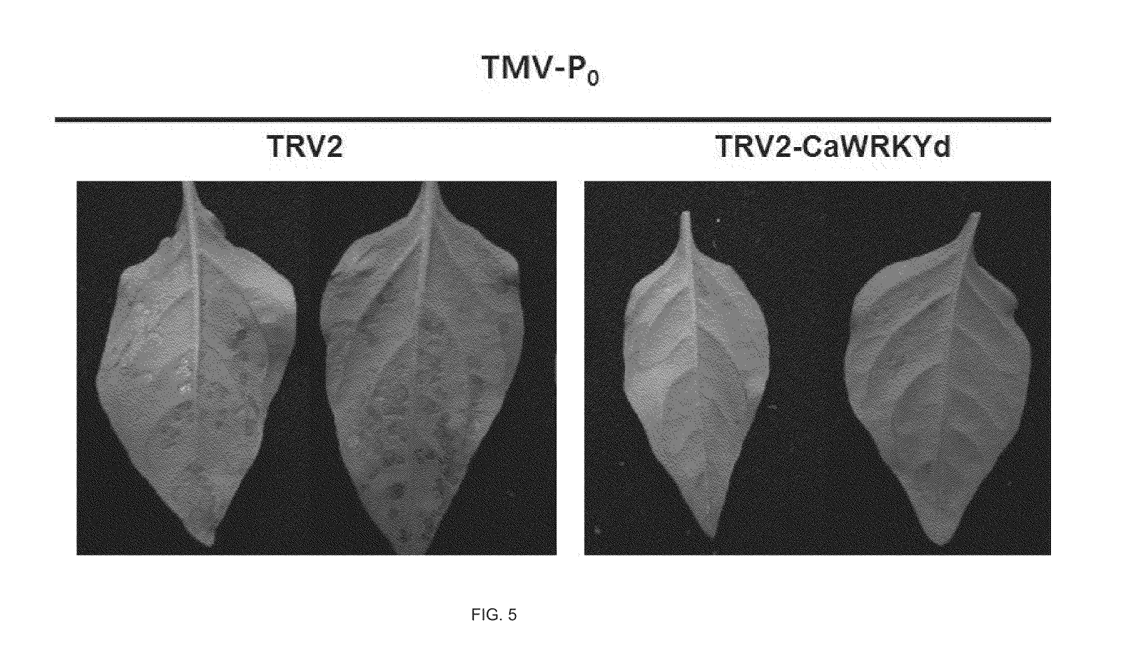

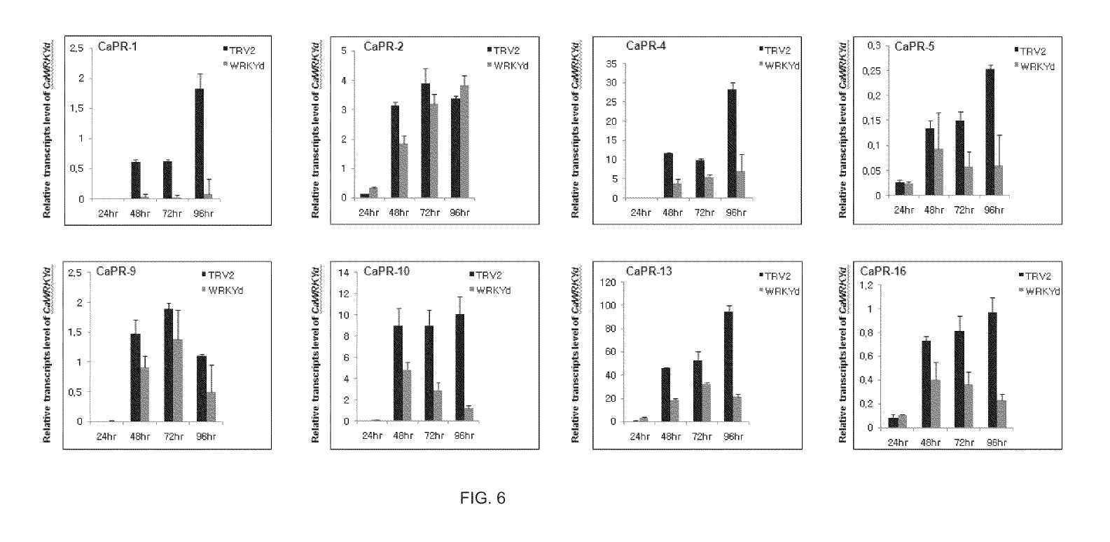

During an incompatible interaction with TMV-P₀, CaWRKYd-knockdown hot pepper plants established by virus inducing gene silencing (VIGS) (Chung, E., Seong, E., Kim, Y.-C., Eun Joo Chung, E. J., Oh, S.-K., Lee, S., Park, J. M., Joung, Y. H., and Choi, D.(2004) A method of high frequency virus-induced gene silencing in chili pepper (Capsicum annuum L. cv. Bukang; Mol. Cells 17: 377-380.; Ratcliff, F., Martin-Hernandez, A. M. and Baulcombe, D. C. (2001) Tobacco rattle virus as a vector analysis of gene function by silencing. Plant J. 25, 237-245) were monitored to examine gene expression pattern. Knockdown of CaWRKYd expression in pepper plants resulted in decreasing TMV-P₀-triggered hypersensitive cell death. In addition, the expression levels of PR genes in the knockdown plants decreased during interactions with virus (FIGS. 5 and 6).

Also, the present invention pertains to an expression vector carrying the gene, a plant cell transformed therewith, and a transgenic plant containing the same.

The present invention provides an expression vector carrying the gene. The gene to be inserted into the expression vector contains a nucleotide sequence encoding CaWRKYd and preferably a nucleotide sequence of SEQ ID NO. 1.

So long as it is used as an expression vector for plant transformation, any vector may be employed in the present invention to insert the CaWRKYd gene into plants. For example, there is a pCAMBIA2300 vector or a pCAMBIA2300 M2 HA2 vector.

The present invention also provides a transformed plant cell anchoring the gene therein. The type of plant cell into which the vector of the present invention is introduced is not limited to a particular form so long as it can regenerate into a plant. Examples of the plant cell include cultured cell suspensions, protoplasts, leaf sections and calluses. The expression vector may be introduced into plant cells using a well-known method such as a polyethylene glycol method, electroporation, Agrobacterium mediation or particle bombardment.

The present invention provides a transgenic plant with improved disease resistance, differentiated from the plant cell by tissue culturing. The differentiation via tissue culturing to obtain the transgenic plant of the present invention may be preferably conducted using a typical method.

These results indicate that CaWRKYd according to the present invention can function as an important transcription factor and are involved in crosstalk between endogenous hormone signaling transductions. Also, CaWRKYd according to the present invention is involved in plant defense responses, possibly through a CaMK1 cascade induced by TMV-P₀, and acts as a positive regulator that imparts disease resistance to plants.

A better understanding of the present invention may be obtained through the following examples which are set forth to illustrate, but are not to be construed as limiting the present invention.

### Example 1

### Yeast Two-Hybrid Analysis

A MatchMaker yeast two-hybrid system was purchased from Clontech. CaMK1 and CaWRKYd fragments were cloned into pGBKT7 (DNA binding domain vector) using EcoRI and BamHI. Separately, CaMK1 and CaWRKYd were cloned into pGADT7 (DNA activation domain vector) using EcoRI and BamHI. These cloned vectors were co-transformed into an AH109 strain. The transformed yeast cells were selected on minimal synthetic dropout (SD) medium lacking Leu and Trp. Yeast cells were allowed to grow at 30° C. for 5-7 days and then subjected to X-α-gal assays in which beta-galactosidase activity was monitored using 5-bromo-4-chloro-3-indolyl α-d-galactoside(X-gal) as a substrate.

### Example 2

### DNA Isolation and Sequence Analysis

Sequence analysis showed that CaWRKYd has a single WRKY domain and a Cys₂-His₂ zinc-finger motif and could be assigned to WRKY group IIa. As seen in FIG. 1, the nucleotide sequence of CaWRKYd cDNA is 1681 bp long (SEQ ID NO. 1), and has an open reading frame (ORF) comprised of 963 nucleotides encoding 320 amino acid residues (SEQ ID NO. 2). The amino acid sequence was compared with WRKY sequences derived from various plants (FIGS. 2 and 3).

### Example 3

### Plant Cultivation and CaWRKYd Expression in Response to Pathogen Inoculation

A hot pepper plant (Capsicum annuum L. cv. Bugang), which is resistant to the TMV-P₀ pathotype but susceptible to the P_{1.2} pathothype of PMMoV, was used as a plant material. Plants were grown at 23° C. in a growth chamber with a 16 hrs light/8 hrs dark photo period cycle. Healthy and well-expanded leaves from 2-month-old plants were used for pathogen inoculation and nucleic acid extraction. TMV-P₀ or PMMoV-P_{1.2} strains were maintained in infected leaves of tobacco (Nicotiana tabacum cv. Samsun) in CaCl₂-containing petri dishes. Leaf sap containing TMV-P₀ or PMMoV-P_{1.2} was prepared by grinding the infected leaves in 0.25 M phosphate buffer containing 5 mM EDTA. To inoculate plants, sap containing the virus was applied to the surface of the 4^{th} or 5^{th} fully expanded leaf of the hot pepper plants and rubbed with carborundum (mesh 500) (Hayashi Chemical, Japan). Mock-inoculated plants were rubbed with phosphate buffer and carborundum only. To monitor systemic responses, leaves two behind the inoculated leaves were inoculated with TMV-P₀ sap, and upper non-inoculated leaves were harvested at 0, 6, 12, 24, 48, and 72 hrs after inoculation (DAI).

Expression patterns of CaWRKYd upon inoculation with TMV-P₀ were analyzed by qRT-PCR and the results are shown in FIG. 4.

In this context, RNA extraction, RT-PCR and quantitative real-time PCR analysis were performed as follows. First, 50 mg of the plant material was ground using mortar and pestle in liquid nitrogen to yield a whitish powder. This powder was transferred to a 1.5 mL e-tube containing 0.5 mL of RNA extraction buffer (0.1 M Tris-HCl, pH 8.0, 0.1 M LiCl, 10 mM EDTA, and 1% SDS) and water-saturated phenol (pH 4.5) and then vigorously vortexed. An equal volume of chloroform was added to the tube and then vortexed vigorously, followed by centrifugation at 4° C. for 10 min at 13,000 rpm. The supernatant was transferred to a new tube to which 0.5 mL of chloroform was then added before vigorous vortexing. Again, the tube was centrifuged at 13,000 rpm for 10 min at 4° C. The supernatant was transferred to a new tube and 0.5 mL of 4 M LiCl2 was added, mixed by gentle inversion, and incubated overnight at 80° C. After centrifugation at 13,000 rpm for 30 min at 4° C., the supernatant was removed and the pellet was washed with 70% ethanol and dissolved in DEPC-treated water.

The total RNA thus extracted was used for RT-PCR analysis. Five micrograms of each RNA sample was reverse transcribed using MMLV-reverse transcriptase (Promega, USA). Each of the first-strand cDNA products was amplified by PCR using primers for CaWRKYd 3″-UTR, CaPR-1, -2, -4, -5, -9, -10, -13, -16, actin, and PIN2 (Table 1, SEQ ID NOS. 3 to 22). Real-time PCR was performed using a KAPA SYBR FAST qPCR Kit (KAPABIOSYSTEM) and gene-specific primers in a LightCycler 480 (Roche, Germany). Combinations of qRT-PCR primers used for specific mRNA detection are given in Table 1.

TABLE 1

| Gene name | Sequence(5′ to 3′) |
|---|---|
| CaWRKYd 3′-UTR | CAAGTTTTCAAGCAGCCTTAG |
| | (SEQ ID NO. 3) |
| | CTTGACAACATATCCTAGTGGCTA |
| | (SEQ ID NO. 4) |
| CaPR-1 qRT-PCR | GAGGACAACGTCCGTATGGT |
| | (SEQ ID NO. 5) |
| | AACTCCAGTTACTGCACCATTAGA |
| | (SEQ ID NO. 6) |
| CaPR-2 qRT-PCR | CTACTTAAGCTTTGCAAGACACCA |
| | (SEQ ID NO. 7) |
| | AGATCTCTTTCCTCATCGTCACTT |
| | (SEQ ID NO. 8) |
| CaPR-4 qRT-PCR | GAACACAAGCAACGGTGAGA |
| | (SEQ ID NO. 9) |
| | GGCACTTGTTTAGGCAGAGC |
| | (SEQ ID NO. 10) |
| CaPR-5 qRT-PCR | TTGCCAAAGTTGGACTACTGATTA |
| | (SEQ ID NO. 11) |
| | TCAACCAAATTGAACAAAAAGAGA |
| | (SEQ ID NO. 12) |
| CaPR-9 qRT-PCR | GACTAGTTTCAAGAGCATCA |
| | (SEQ ID NO. 13) |
| | AATTGTATAGCCTGTAGCTG |
| | (SEQ ID NO. 14) |
| CaPR-10 qRT-PCR | CTGTCTATGTTTAAGCCAATGACC |
| | (SEQ ID NO. 15) |
| | AAAGTTCTTTCCATGACAACCAAT |
| | (SEQ ID NO. 16) |
| CaPR-13 qRT-PCR | CTGTAAAGAGCTCACAAAAC |
| | (SEQ ID NO. 17) |
| | GCGAGAGTTTTAGCATTAC |
| | (SEQ ID NO. 18) |
| CaPR-16 qRT-PCR | CCCTAGAGGACTTGTACATT |
| | (SEQ ID NO. 19) |
| | TTAGTCAAGACAGAATCAGG |
| | (SEQ ID NO. 20) |
| CaActin qRT-PCR | CTTGTCTGTGATAATGGAAC |
| | (SEQ ID NO. 21) |
| | GGATACTTCAAGGTGAAGAAT |
| | (SEQ ID NO. 22) |

CaWRKYd 3-UTR: primers used to confirm the knock-down of CaWRKYd in the CaWRKYd-silenced pepper plants

CaPR-1/-2/-3/-4/-5/-9/-10/-13/-16: primers for amplifying various pathogenesis-related (PR) genes present in hot pepper to examine the expression patterns of various pathogenesis-related (PR) genes in control and CaWRKYd-silenced plants

CaActin: primers for amplifying the house keeping gene present in hot pepper, used as a control for comparison of cDNA in qRT-PCR

### Example 4

### CaWRKYd Expression According to Chemical and Wounding Treatment

Two-month-old pepper leaves were sprayed with 10 mM SA solution. Another set of control plants, whenever possible, was similarly treated with distilled water. The leaves were harvested at predetermined time points after treatment, quickly frozen in liquid nitrogen and stored at −80° C. For the treatment with 10 mM ethephon or 100 μM methyl jasmonate (MeJA), unrooted pepper plants were placed for various time periods in Falcon tubes filled with ethephon, MeJA or distilled water, and then frozen in liquid nitrogen for additional analysis. For the wounding treatment, the leaves were incised with a pair of scissors. The wounded leaves were harvested at 0, 1, 2, 6, 12, 24 and 48 hrs after treatment.

The induction of Capsicum annuum CaWRKYd by defense signaling molecules and wounding treatments was analyzed using qRT-PCR as described in Example 2 and the results are shown in FIGS. 7A to 7D. FIG. 7A shows that transcripts of CaWRKYd were detected in chili pepper plants (Capsicum annuum cv. Bukang) treated with salicylic acid (SA). Hot pepper leaves were sprayed with 10 mM SA or mock-treated with 10% ethanol. FIG. 7B shows that transcripts of CaWRKYd were detected in chili pepper plants (Capsicum annuum cv. Bukang) treated with ethephon. Hot pepper leaves were sprayed with 10 mM ethephon or mock-treated with 10% ethanol. FIG. 7C shows that transcripts of CaWRKYd were detected in chili pepper plants (Capsicum annuum cv. Bukang) treated with MeJA. Hot pepper leaves were sprayed with 100 μM MeJA or mock-treated (10% ethanol). FIG. 7D shows that transcripts of CaWRKYd were detected in chili pepper plants (Capsicum annuum cv. Bukang) wounded at various time-points. Wounding was applied to the leaves of chili pepper plants by crushing the apical lamina several times, resulting in effectively wounding ˜90% of the leaf area. Values indicate mRNA levels normalized to the expression of the CaActin gene

### Example 5

### Analysis of CaWRKYd Function Using VIGS (Virus-Induced Gene Silencing)

The PCR-amplified, 216-bp 3″UTR fragment of CaWRKYd was cloned into the BamHI site of the TRV2 vector (Liu et al., 2002; Chung et al., 2004). PCR primers had the sequences of 5″-GGATCCAAATGTTCCAGAAAAAG-3″ (SEQ ID NO. 23) and 5″-GGATCCAGGCCCACCCAAGTCAC-3″ (SEQ ID NO. 24). pTRV2 and pTRV2-CaWRKYd were transformed into the Agrobacterium tumefaciens strain GV3101 and the transformed cells were selected on YEP media containing 50 mg kanamycin and 100 mg rifampicin. The transformants were grown at 28° C. Following centrifugation, the bacteria was resuspended in buffer containing 10 mM MES-KOH (pH 5.7), 10 mM MgSO₄, and 200 μM acetosyringone to A₆₀₀ of 0.3 and allowed to stand at room temperature for 2˜4 hrs. Cultures of the transformants were mixed at a 1:1 ratio with A. tumifaciens anchoring the TRV1 vector thereat (OD600 nm of 0.2), and the mixture was infiltrated into Bukang Chili hot pepper plants. For each experiment, 50 chili pepper plants were inoculated with the pTRV2 vector or the pTRV2-CaWRKYd-3″ UTR vector respectively. One month after this inoculation, CaWRKYd-silenced plants were allowed to be challenged with TMV-P₀. The leaves were used for RNA extraction.

Hydrogen peroxide accumulation was visualized with 3,3′-diaminobenzidine (DAB) as described in Thordal-Christensen et al. (1997) with some modification. Leaves excised from TRV2 and TRV2-CaWRKYd 3′UTR-silenced plants were placed in 50 mL Falcon tubes containing a DAB solution (1 mg ml⁻¹). The DAB solution was infiltrated into leaves twice for 10 min in a vacuum. Then, they were transferred to 96% (v/v) ethanol and chlorophyll was removed by boiling the 96% (v/v) ethanol for 10˜30 min. DAB was rapidly absorbed by the plants and polymerized locally in the presence of H₂O₂ and peroxidase, giving a visible brown stain.

FIG. 5 is of photographs showing the silencing of the CaWRKYd gene in chili pepper (Capsicum annuum) plants. Two-week-old chili pepper seedlings were inoculated with the pTRV1 and pTRV2-CaWRKYd vectors by agroinfiltration. Four weeks later, upper leaves were infected with TMV-P₀. The photographs show the leaves of pTRV2 and pTRV2-CaWRKYd 4 days after inoculation with TMV-P₀.

FIG. 6 shows expression patterns of the Capsicum annuum pathogenesis-related (CaPR) genes in CaWRKYd-silenced plants upon inoculation with TMV-P₀. The expression of CaPR genes in the leaves of pTRV2 and pTRV2-CaWRKYd silenced plants was analyzed by qRT-PCR as suggested in Example 2. Total RNA was extracted from leaves 24, 48, 72 and 96 hrs after TMV-P₀ inoculation. Values indicate mRNA levels normalized to the expression of the CaActin gene.

### Example 6

### Expression Pattern of CaWRKYd According to Organ

The tissue-specific expression patterns of CaWRKYd in various organs of Chili hot pepper plants were analyzed using qRT-PCR. Total RNA isolated from various tissues as described in Example 2 was used. As seen in FIG. 8, CaWRKYd was expressed in all organs including the leaves, roots, stems, flowers and fruits, especially at high levels in the leaves.

### Example 7

### Subcellular Localization of CaWRKYd

The subcellular localization of CaWRKYd was determined using the 35S-GFP3G-CaWRKYd-fusion protein. A full-length CaWRKYd open reading frame (ORF) without the termination codon was prepared by PCR using CaWRKYd cDNA as a template in the presence of a pair of primers (forward, 5′-CCCGGGCAATGCCTGACAAA-3′ (SEQ ID NO. 25) and reverse, 5′-CCCGGGTTTGTCTGTATGATTA-3′ (SEQ ID NO. 26)). PCR products containing an XmaI site were digested with XmaI and fused into a GFP expression factor. The N-terminal region of the PCR-amplified CaWRKYd cDNA fragment was fused to the C-terminal region of the GFP3G expression vector. A 35S-GFP3G vector without CaWRKYd was used as a control. A plasmid DNA of the appropriate fusion construct (2˜3 μg each of 35S-GFP3G-CaWRKYd or p35S-GFP3G) was introduced into Arabidopsis protoplasts using PEG-mediated transformation. Fluorescent images were captured using a UV light incident fluorescence microscope (Zeiss, Axioskop, Germany) equipped with a fluorescein isothiocyanate filters (an excitation filter of 520 nm and an emission filter of 488). The results are shown in FIGS. 9A to 9B.

FIG. 9A is a schematic view showing the structure of the GFP3G::CaWRKYd fusion gene. FIG. 9B shows the subcellular localization of the GFP3G::CaWRKYd fusion protein. The expression of the introduced genes was monitored 24 hrs after introduction using fluorescence or light microscopy.

As described hitherto, the hot pepper-derived novel gene CaWRKYd is provided whose expression is induced by the infection of TMV-P₀, and treatment with SA, MeJA, ethephon, and wound infliction. It is identified to participate in plant defense responses. Therefore, the gene allows the production of plants resistant to diseases and can be very effectively used in studying defense response mechanisms of plants.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An isolated gene encoding a hot pepper-derived stress-resistant transcription factor having an amino acid sequence of SEQ ID NO. 2.

2. The isolated gene of claim 1, wherein the gene has a nucleotide sequence of SEQ ID NO. 1.

3. An isolated protein having an amino acid sequence of SEQ ID NO. 2.

4. A recombinant vector carrying the gene of claim 1.

5. A transgenic plant anchoring the gene of claim 1 therein.

6. A transgenic plant anchoring the recombinant vector of claim 4 therein.

7. A pTRV2-CaWRKYd vector for VIGS (virus induced gene silencing), constructed by cloning a hot pepper transcription factor gene CaWRKYd having a nucleotide sequence of SEQ ID NO. 1 into a TRV vector.

8. A transgenic plant in which a CaWRKYd gene is down-regulated, the transgenic plant anchoring a pTRV2-CaWRKYd vector for VIGS (virus induced gene silencing) which is constructed by cloning a hot pepper transcription factor gene CaWRKYd having a nucleotide sequence of SEQ ID NO. 1 into a TRV vector.
